(19) United States
(12) Patent Application Publication
(10) Pub. No.: US 2012/0329905 A1
(43) Pub. Date: Dec. 27, 2012

(54) High Refractive Index Lenses

(76) Inventors: Ivan M. Nunez, Penfield, NY (US); Jay F. Kunzler, Canandaigua, NY (US); Jennifer Hunt, Batavia, NY (US); Joseph A. McGee, Canandaigua, NY (US)

(21) Appl. No.: 13/483,397

(22) Filed: May 30, 2012

Related U.S. Application Data
(60) Provisional application No. 61/500,699, filed on Jun. 24, 2011.

(51) Int. Cl. C08G 63/688; C07C 333/20; C07C 333/04; C08L 81/00

(52) U.S. Cl. 523/113; 558/240; 528/360

(57) ABSTRACT

An optical copolymer comprising: a constituent derived from a sulfated lens monomer of general formula I

[PG-R¹—NHC(Y)S—R²]ₙ—R³ I

PG is a polymerizable group selected from the group consisting of vinyl, acryloyl, acryoyloxy, methacryoyl and methacryoyloxy; acrylamido and methacrylamido; Y is oxygen or sulfur; and n is 2, 3 or 4. Also, R¹ is an organic group of one to eight carbon atoms and optionally with one or more oxygen atoms or an aromatic ring. R² is a bond or an organic group of one to six carbon atoms. R³ is a bond, an organic group of one to ten carbons atoms, carbon, oxygen, sulfur or a disulfide; and a lens monomer. The sulfated lens monomer is derived from a first component of formula, PG-R¹—NC(Y), and a polythiol of formula, R³—[R²—SH]ₙ.

### CROSS REFERENCE

This application claims the benefit of Provisional Patent Application No. 61/500,699 filed Jun. 24, 2011, which is incorporated by reference herein.

### FIELD OF THE INVENTION

The invention relates to optical copolymers and to optical lenses made from such copolymers.

### BACKGROUND OF THE INVENTION

Since the 1940s intraocular lenses (IOLs) have been utilized as replacements for diseased or damaged natural ocular lenses. In most cases, an IOL is implanted within an eye at the time of surgically removing the diseased or damaged natural lens, such as for example, in the case of cataracts. For decades, the preferred material for fabricating IOLs was poly(methyl methacrylate), which is a rigid, glassy polymer.

As the technology in IOL materials progressed, softer, more flexible IOLs have gained acceptance because of their ability to be compressed, folded, rolled or otherwise deformed. As a result, the IOLs can be deformed prior to insertion of the lens through an incision in the cornea and into the eye. Once inserted, the IOL is carefully unfolded by the surgeon thereby returning the lens to its original pre-deformed shape. These softer IOLs can be inserted into an incision of less than 3.0 mm, whereas the earlier, more rigid IOLs required an incision size of 5 to 7.0 mm, i.e., an incision size slightly larger than the diameter of the optic portion of the IOL. Since larger incisions lead to an increased incidence of postoperative complications, the softer, more flexible IOLs are typically preferred by ocular surgeons.

The refractive power of a lens is a function of its shape and the refractive index of the material of which it is made. Accordingly, a lens made from a material having a higher refractive index can be thinner and provide the same refractive power as a lens made from a material having a relatively lower refractive index. For IOLs designed to be rolled or folded for insertion through a small incision, a lens of thinner cross section is inherently more flexible and can be rolled or folded to a smaller cross section.

The size and mechanical characteristics of the deformable IOLs play an important role. As is well understood by those skilled in the art, for successful implantation, the deformable IOL must have sufficient structural integrity, elasticity and elongation and be small enough in size to permit deforming for insertion through a small incision. After insertion, the lens must, of course, regain its original shape and have sufficient structural integrity to retain such shape under normal use conditions.

In general, the materials of current commercial IOLs fall into one of three general categories: silicones, low-water hydrophilic acrylics and hydrophobic acrylics. Hydrophobic acrylic materials with a relatively low glass transition temperature (Tg° C.) possess important material characteristics—they typically have a high refractive index and unfold with a greater degree of control.

### SUMMARY OF THE INVENTION

A sulfated lens monomer of formula I.

[PG-R¹—NHC(Y)S—R²]ₙ—R³ I

PG is a polymerizable group selected from the group consisting of vinyl, acryloyl, acryoyloxy, methacryoyl and methacryoyloxy; acrylamido and methacrylamido; Y is oxygen or sulfur; and n is 2, 3 or 4. Also, R¹ is an organic group of one to eight carbon atoms and optionally with one or more oxygen atoms or an aromatic ring. R² is a bond or an organic group of one to six carbon atoms. R³ is a bond, an organic group of one to ten carbons atoms, carbon, oxygen, sulfur or a disulfide.

An optical copolymer comprising: a constituent derived from lens monomer of general formula I as defined above. The lens monomer is derived from a first component of formula, PG-R¹—NC(Y), and a polythiol of formula, R³—[R²—SH]ₙ.

An optical copolymer will comprise a constituent derived from sulfated lens monomer of formula I as defined above, and a constituent derived from an aromatic monomeric component, the homopolymer of which has a refractive index of at least about 1.48.

An optical copolymer can also be described as comprising a first constituent derived from a first component of formula III.

PG-R—NCY III

Again, PG is a polymerizable group selected from the group consisting of vinyl, acryloyl, acryoyloxy, methacryoyl and methacryoyloxy; acrylamido and methacrylamido; R is an organic group of one to eight carbon atoms and optionally with one or more oxygen atoms or an aromatic ring; and Y is oxygen or sulfur. The optical copolymer will also include a second constituent derived from a polythiol, and a third constituent derived from an aromatic monomeric component, the homopolymer of which has a refractive index of at least about 1.48.

### BRIEF DESCRIPTION OF THE DRAWINGS

FIG. 1 is a front elevational view of an accommodating intraocular lens;

FIG. 2 is a side view of the FIG. 1 lens;

FIG. 3 is a detail view of a hinge of the FIG. 1 lens;

FIG. 4 is an elevational view of an accommodating intraocular lens;

FIG. 5 is a sectional view of the lens of FIG. 1 disposed in an eye, showing the lens optic in a generally anterior position and in a posteriorly vaulted position;

FIG. 6 is an elevational view of another accommodating intraocular lens;

FIG. 7 is an elevational view of another accommodating intraocular lens; and

FIG. 8 is an elevational view of another accommodating intraocular lens wherein a generally annular glare-reducing component is disposed about an optic.

### DETAILED DESCRIPTION OF THE INVENTION

The invention is directed to an optical copolymer comprising units of a sulfated lens monomer, which is prepared by the reaction of an organic isocyanate, or an organic isothiocyanate, and a polythiol, and units of lens monomer. The optical copolymer can also include units of hydrophilic monomer as well as elastic-modifying monomer to optimize the material properties required of an ophthalmic lens, and particularly an intraocular lens (IOL). The optical copolymer will possess the mechanical properties required of an intraocular lens that is to be inserted into a lens capsule of a patient through a small surgical incision of less than 2.5 mm, preferably less than 2.0 mm, by folding the IOL in an intraocular lens inserter device. For example, the optical copolymer will have a refractive index of at least 1.48 and a modulus of elasticity of from 500 MPa to 10,000 MPa.

As stated, IOLs are designed to be rolled or folded for insertion through a small surgical incision. An IOL of thinner cross section is inherently more flexible and can be rolled or folded to a smaller cross section, and thereby fitted through an ever smaller incision. However, as one reduces or thins the cross section of the optic portion of a lens the total refractive power of the lens is reduced for any one given material. To compensate for the thin optics and the loss of refractive power one can use a material with a greater refractive index. Accordingly, the IOLs that are prepared by the materials described will be optically clear and have a relatively high refractive index, for example, at least 1.48, 1.50 or 1.52. The IOLs will tend to have an optic cross section of from 0.5 mm to 2.0 mm. As nearly all IOLs are designed with a double convex optic the optic cross section is measured from a central anterior surface to a central posterior surface of the lens. In most cases, the measurement is made along the central optic axis.

In one embodiment, the invention is directed to a sulfated lens monomer of formula I.

[PG-R¹—NHC(Y)S—R²]ₙ—R³ I

PG is a polymerizable group selected from the group consisting of vinyl, acryloyl, acryoyloxy, methacryoyl and methacryoyloxy; acrylamido and methacrylamido; Y is oxygen or sulfur; and n is 2, 3 or 4. Also, R¹ is an organic group of one to eight carbon atoms and optionally with one or more oxygen atoms or an aromatic ring. R² is a bond or an organic group of one to six carbon atoms. R³ is a bond, an organic group of one to ten carbons atoms, carbon, oxygen, sulfur or a disulfide. The term “organic group” is a carbon-based group, typically with pendent hydrogen, e.g., an alkyl or cyclic alkyl. Of course, it is understood by one of ordinary skill that common substituents can substitute for the hydrogen, e.g., fluorine.

The sulfated lens monomer is prepared from an isocyanate of thioisocyanate of formula, PG-R¹—NC(Y) and a polythiol of formula, R³—[R²—SH]ₙ. PG, R¹, R², R³ and Y are as defined by formula I. In one instance, the sulfated lens monomer of formula I will have R³ as oxygen, sulfur or disulfide and R² as an alkylene with two to four carbons or R² will comprise a phenyl group. In another instance, the sulfated lens monomer of formula I will have R³ as a bond, a carbon or —CH and R² is alkylene with one to three carbons. To provide additional control of refractive index R¹ can include an aromatic ring system. In another instance, R¹ is a straight or branched alkylene radical with an optional heteroatom along the alkylene chain, e.g., oxygen or sulfur. At times, the term “iso(thio)cyanate” is used and refers to either the isocyanate or the corresponding isothiocyanate analog.

Other sulfated lens monomer of formula I where PG, R¹, R², R³ and Y are as defined include R¹ is an alkylene with one to three carbons or R¹ comprises a phenyl group. In another instance R¹ includes an aromatic ring; R² is alkylene with two to four carbons; R³ is carbon, ethyl or propyl; and Y is oxygen.

A particular sulfated lens monomer is of formula II

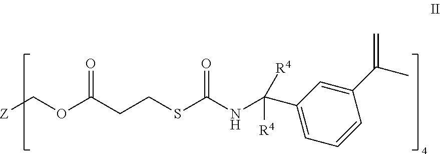

wherein Z is carbon, or an alkyl with two to four carbons and R⁴ is independently selected from hydrogen or methyl.

Exemplary iso(thio)cyanates include

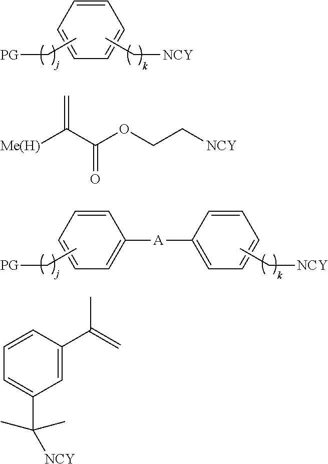

wherein PG and Y are as defined for general formula I, A is Y or a disulfide; and j is 1, 2 or 3 and k is 0, 1, 2 or 3.

In one embodiment, an optical copolymer of the invention will comprise a constituent derived from sulfated lens monomer of formula I as defined above, and a constituent derived from an aromatic monomeric component, the homopolymer of which has a refractive index of at least about 1.48.

Rather than separately preparing a sulfated lens monomer, and then reacting the sulfated lens monomer with other lens monomers, infra, or hydrophilic monomers, infra, an optical copolymer of the invention can be described as comprising a first constituent derived from a first component of formula III.

PG-R—NCY III

Again, PG is a polymerizable group selected from the group consisting of vinyl, acryloyl, acryoyloxy, methacryoyl and methacryoyloxy; acrylamido and methacrylamido; R is an organic group of one to eight carbon atoms and optionally with one or more oxygen atoms or an aromatic ring; and Y is oxygen or sulfur. The optical copolymer will also include a second constituent derived from a polythiol, and a third constituent derived from an aromatic monomeric component, the homopolymer of which has a refractive index of at least about 1.48.

The optical copolymer is optically clear, and will preferably possess a refractive index of at least about 1.51. The term “optically clear” refers to a polymeric material with a transmissibility from 500 nm to 800 nm of at least 85%. An exemplary list of aromatic monomeric component includes styrene, vinyl carbazole, vinyl naphthalene, benzyl acrylate, phenyl acrylate, naphthyl acrylate, pentabromophenyl acrylate, 2-phenoxyethyl acrylate, 2-phenoxyethyl methacrylate and any one mixture thereof.

The organic isocyanate or organic isothiocyanate of choice is then reacted with a polythiol of choice to provide the sulfated-prepolymer. As indicated by the relative long list of exemplary polythiols that follow essentially any polythiol can be used to form the described sulfated prepolymer including aliphatic polythiols such as methanedithiol, 1,2-ethanedithiol, 1,2-propanedithiol, 1,3-propanedithiol, 1,6-hexanedithiol, 1,2,3-propanetrithiol, 1,2-cyclohexanedithiol, 2,2-dimethylpropane-1,3-dithiol, 2,3-dimercaptosuccinic acid 2-mercaptoethyl ester, 2,3-dimercapto-1-propanol 2-mercaptoacetate, 2,3-dimercapto-1-propanol 3-mercaptopropionate, diethylene glycol bis(2-mercaptoacetate), diethylene glycol bis(3-mercaptopropionate), 1,2-dimercaptopropyl methyl ether, 2,3-dimercaptopropyl methyl ether, 2,2-bis(mercaptomethyl)-1,3-propanedithiol, bis(2-mercaptoethyl)ether, ethylene glycol bis(2-mercaptoacetate), ethylene glycol bis(3-mercaptopropionate), trimethylolpropane bis(2-mercaptoacetate), trimethylolpropane bis(3-mercaptopropionate), pentaerythritol tetrakis(2-mercaptoacetate) and pentaerythritol tetrakis(3-mercaptopropionate); as well as aromatic polythiols such as 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(mercaptoethyl)benzene, 1,3-bis(mercaptoethyl)benzene, 1,4-bis(mercaptoethyl)benzene, 1,2-bis(mercaptomethyleneoxy)benzene, 1,3-bis(mercaptomethyleneoxy)benzene, 1,4-bis(mercaptomethyleneoxy)benzene, 1,2-bis(mercaptoethyleneoxy)benzene, 1,3-bis(mercaptoethylenoxy)benzene, 1,4-bis(mercaptoethyleneoxy)benzene, 1,2,3-trimercaptobenzene, 1,2,4-trimercaptobenzene, 1,3,5-trimercaptobenzene, 1,2,3-tris(mdcaptomethyl)benzene, 1,2,4-tris(mercaptomethyl)benzene, 1,3,5-tris(mercaptomethyl)benzene, 1,2,3-tris(mercaptoethyl)benzene, 1,2,4-tris(mercaptoethyl)benzene, 1,3,5-tris(mercaptoethyl)benzene, 1,2,3-tris(mercaptomethyleneoxy)benzene, 1,2,4-tris(mercaptomethyleneoxy)benzene, 1,3,5-tris(mercaptomethyleneoxy)benzene, 1,2,3-tris(mercaptoethyleneoxy)benzene, 1,2,4-tris(mercaptoethyleneoxy)benzene, 1,3,5-tris(mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(mercaptoethyl)benzene, 1,2,3,5-tetrakis(mercaptoethyl)benzene, 1,2,4,5-tetrakis(mercaptoethyl)benzene, 1,2,3,4-tetrakis(mercaptomethyleneoxy)benzene, 1,2,3,5-tetrakis(mercaptomethyleneoxy)benzene, 1,2,4,5-tetrakis(mercaptomethyleneoxy)benzene, 1,2,3,4-tetrakis(mercaptoethyleneoxy)benzene, 1,2,3,5-tetrakis(mercaptoethyleneoxy)benzene, 1,2,4,5-tetrakis(mercaptoethyleneoxy)benzene, 2,2′-dimercaptobiphenyl, 4,4′-dimercaptobiphenyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-dimethylbenzene-1,3-dithiol, 9,10-anthracenedimethanethiol, 1,3-di(p-methoxyphenyl)propane-2,2-dithiol, 1,3-diphenylpropane-2,2-dithiol, phenylmethane-1,1-dithiol and 2,4-di(p-mercaptophenyl)pentane; heterocyclic polythiols such as 2-methylamino-3,6-dithiol-sym-triazine, 2-ethylamino-4,6-dithiol-sym-triazine, 2-amino-4,6-dithiol-sym-triazine, 2-morpholino-4,6-dithiol-symtriazine, 2-cyclohexylamino-4,6-dithiol-sym-triazine, 2-methoxy-4,6-dithiol-sym-triazine, 2-phenoxy-4,6-dithiol-sym-triazine, 2-thiobenzeneoxy-4,6-dithiolsym-triazine and 2-thiobutyloxy-4,6-dithiol-sym-triazine; aromatic polythiols containing sulfur atoms in addition to mercapto groups such as 1,2-bis(mercaptomethylthio)benzene, 1,3-bis(mercaptomethylthio)benzene, 1,4-bis(mercaptomethylthio)benzene, 1,2-bis(mercaptoethylthio)benzene, 1,3-bis(mercaptoethylthio)benzene, 1,4-bis(mercaptoethylthio)benzene, 1,2,3-tris(mercaptomethylthio)benzene, 1,2,4-tris(mercaptomethylthio)benzene, 1,3,5-tris(mercaptomethylthio)benzene, 1,2,3-tris(mercaptoethylthio)benzene, 1,2,4-tris(mercaptoethylthio)benzene 1,3,5-tris(mercaptoethylthio)benzene, 1,2,3,4-tetrakis(mercaptomethylthio)benzene, 1,2,3,5-tetrakis(mercaptomethylthio) benzene, 1,2,4,5-tetrakis(mercaptomethylthio)benzene, 1,2,3,4-tetrakis(mercaptoethylthio) benzene, 1,2,3,5-tetrakis(mercaptoethylthio)benzene, 1,2,4,5-tetrakis(mercaptoethylthio) benzene and aromatic ring alkylated derivatives of these polythiols; aliphatic polythiols containing sulfur atoms in addition to mercapto groups such as bis(mercaptomethyl) sulfide, bis(mercaptoethyl) sulfide, bis(mercaptopropyl) sulfide, bis(mercaptomethylthio)methane, bis(2-mercaptoethylthio)methane, bis(3-mercaptopropyl)methane, 1,2-bis(mercaptomethylthio)ethane, 1,2-bis(2-mercaptoethylthio)ethane, 1,2-bis(3-mercaptopropyl)ethane, 1,3-bis(mercaptomethylthio)propane, 1,3-bis(2-mercaptoethylthio)propane, 1,3-bis(3-mercaptopropylthio)propane, 1,2,3-tris(mercaptomethylthio)propane, 1,2,3-tris(2-mercaptoethylthio)propane, 1,2,3-tris(3-mercaptopropylthio)propane, tetrakis(mercaptomethylthiomethyl)methane, tetrakis(2-mercaptoethylthiomethyl)methane, tetrakis(3-mercaptopropylthiomethyl)methane, bis(2,3-dimercaptopropyl) sulfide, 2,5-dimercapto-1,4-dithian, bis(mercaptomethyl)disulfide, bis(mercaptoethyl)disulfide and bis(mercaptopropyl)disulfide; esters of mercaptoacetic acid, mercaptopropionic acid and mercaptobutyric acid with sulfur containing compounds such as hydroxymethyl sulfide bis(2-mercaptoacetate), hydroxymethyl sulfide bis(3-mercaptopropionate), hydroxyethyl sulfide bis(2-mercaptoacetate), hydroxyethyl sulfide bis(3-mercaptopropionate), hydroxypropyl sulfide bis(2-mercaptoacetate), hydroxypropyl sulfide bis(3-mercaptopropionate), hydroxymethyl disulfide bis(2-mercaptoacetate), hydroxymethyl disulfide bis(3-mercaptopropionate), hydroxyethyl disulfide bis(2-mercaptoacetate), hydroxyethyl disulfide bis(3-mercaptopropionate), hydroxypropyl disulfide bis(2-mercaptoacetate), hydroxypropyl disulfide bis(3-mercaptopropionate), 2-mercaptoethyl ether bis(2-mercaptoacetate), 2-mercaptoethyl ether bis(3-mercaptopropionate), 1,4-dithian-2,5-diol bis(2-mercaptoacetate), 1,4-dithian-2,5-diol bis(3-mercaptopropionate), thiodiglycolic acid bis(2-mercaptoethyl ester), thiodipropionic acid bis(2-mercaptoethyl ester), 4,4-thiodibutyric acid bis(2-mercaptoethyl ester), dithiodiglycolic acid bis(2-mercaptoethyl ester), dithiodipropionic acid bis(2-mercaptoethyl ester), 4,4-dithiodibutyric acid bis(2-mercaptoethyl ester), thiodiglycolic acid bis(2,3-dimercaptopropyl ester), thiodipropionic acid bis(2,3-dimercaptopropyl ester), dithiodiglycolic acid bis(2,3-dimercaptopropyl ester) and dithiodipropionic acid bis(2,3-dimercaptopropyl ester); and heterocyclic compounds containing sulfur atoms in addition to mercapto groups such as 3,4-thiophene-dithiol and 2,5dimercapto-1,3,4-thiadiazol.

The term “polythiol” is also inclusive of compounds that contain both a mercapto group and a hydroxy group such as mercaptoalkanols, mercaptocycloalkanols and mercapto phenols. Exemplary mercapto/hydroxyl compounds include 2-mercaptoethanol, 3-mercapto-1,2-propanediol, glycerol di(mercaptoacetate), 1-hydroxy-4-mercaptocyclohexane, 2,4-dimercaptophenol, 2-mercaptohydroquinone, 4-mercaptophenol, 3,4-dimercapto-2-propanol, 1,3-dimercapto-2-propanol, 2,3-dimercapto-1propanol, 1,2-dimercapto-1,3-butanediol, pentaerythritol tris(3-mercaptopropionate), pentaerythritol mono(3-mercaptopropionate), pentaerythritol bis(3-mercaptopropionate), pentaerythritol tris(thioglycolate), pentaerythritol tetrakis(3-mercaptopropionate); and mercapto group and sulfur atom containing alkanols and phenols such as hydroxymethyltris(mercaptoethylthiomethyl)methane, 1-hydroxyethylthio-3-mercaptoethylthiobenzene, 4-hydroxy-4′-mercaptodiphenyl sulfone, 2-(2-mercaptoethylthio)ethanol, dihydroxyethyl sulfide mono(3-mercaptopropionate), dimercaptoethane monosalicylate, and hydroxyethylthiomethyl-tris(mercaptoethylthiomethyl)methane.

Some of the more exemplary polythiols and their chemical structures follow.

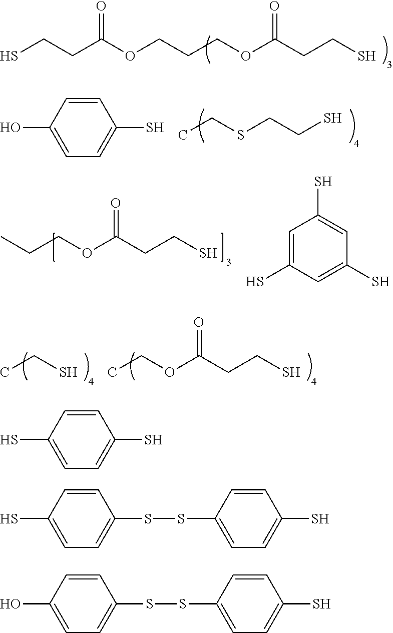

To form the optical copolymer the sulfated lens monomer is polymerized with at least one of: (i) one or more lens monomers (see below); and (ii) one or more hydrophilic monomers. The lens monomer can provide one or more benefits, e.g., enhanced tensile strength, relative to a substantially identical copolymer but without the lens monomer. The lens monomer can also be used to further adjust the requisite properties demanded of a foldable IOL. The hydrophilic monomer can be used to adjust the hydrophilic character of the optical copolymer. In some instances, highly hydrophobic, acrylic polymeric materials can exhibit what is known in the art as glistenings. It is believed that the glistenings are caused by water becoming entrapped within the hydrophobic polymer matrix following implantation of the lens into the eye. The glistenings can interfere with vision, particularly when one is driving an automobile at night. To reduce the probability of glistening, it may prove advantages to have an optical copolymer that can absorb small amounts of water, e.g., from 1.5 wt % to 15 wt %. The term “hydrophilic monomer component” refers to compounds which produce hydrogel-forming homopolymers, that is homopolymers which have an equilibrium water content of at least 25%, based on the weight of the homopolymer, if placed in contact with an aqueous solution for an equilibrated time period.

A particular optical copolymer of interest is prepared from the sulfated lens monomer, PTMC2B, and the polythiol, PETMP. The units of PTMC2B in the copolymer is present from 15 wt. % to 35 wt. %, preferably from 20 wt. % to 28 wt. %, and the units of PETMP from 25 wt. % to 40 wt. % preferably from 30 wt. % to 36 wt. %. The copolymer will also include one or more crosslink agents, and the units derived from such agents are present from 25 wt. % to 50 wt. %, preferably from 35 wt. % to 45 wt. %. In many embodiments of the optical copolymer, the crosslink agents will include polyethylene glycol (PEG) linkages. In most instances, the copolymer is prepared with a photo polymerization process.

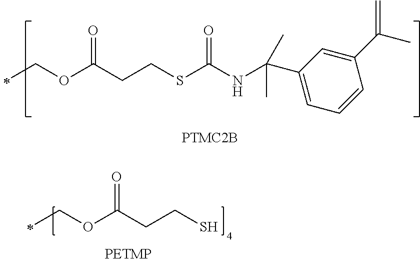

Another optical copolymer of interest is prepared from the sulfated lens monomer, TMPT3P, and the polythiol, TMPTMP. The units of TMPT3P in the copolymer is present from 15 wt. % to 40 wt. %, preferably from 20 wt. % to 28 wt. %, and the units of TMPTMP from 15 wt. % to 40 wt. % preferably from 20 wt. % to 28 wt. %. Again, the copolymer will also include one or more crosslink agents, and the units derived from such agents are present from 25 wt. % to 50 wt. %, preferably from 35 wt. % to 45 wt. %. In many embodiments of the optical copolymer, the crosslink agents will include polyethylene glycol (PEG) linkages. In most instances, the copolymer is prepared with a thermal polymerization process.

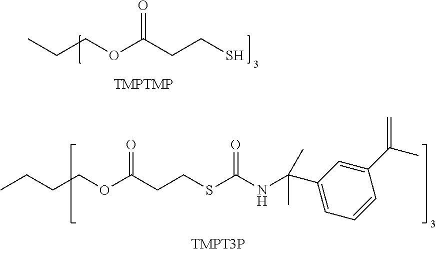

### The Optical Polymeric Materials for Intraocular Lenses

As stated, the optical polymeric materials prepared according to the methods described will include at least one lens monomer including many of that have been identified by those of ordinary skill in art familiar with optical polymeric materials, particularly for ophthalmic devices, e.g., intraocular lenses. Non-limiting examples of such materials include those used in the manufacture of ophthalmic devices, such as siloxy-containing polymers, acrylic, hydrophilic or hydrophobic polymers or copolymers thereof. The optical polymeric materials are of sufficient optical clarity, and will have a relatively high refractive index of approximately 1.48 or greater, preferably 1.50 or greater. Many of these materials are also characterized by a relatively high elongation of approximately 80 percent or greater.

In one embodiment, the optical polymeric materials are prepared as a copolymer from at least two or more monomeric components. The sulfated lens monomer is present in the optical copolymer in an amount of at least 20% by weight, e.g., 20% to 40% by weight, and its homopolymer will have a refractive index of 1.52 or greater, or 1.54 or greater. The term “homopolymer” refers to a polymer that is derived substantially completely from the respective monomeric component. Minor amounts of catalysts, initiators and the like can be included, as is conventionally the case, in order to facilitate the formation of the homopolymer. The other lens monomeric component is present in the copolymer in an amount from 40% to 80% or from 50% to 70%, by weight. Particularly useful lens monomer components include styrene, vinyl carbazole, vinyl naphthalene, benzyl(meth)acrylate, phenyl(meth)acrylate, naphthyl(meth)acrylate, 2-phenoxyethyl(meth)acrylate, 2,3-dibromopropyl-(meth)acrylate, n-butyl(meth)acrylate, n-hexyl(meth)acrylate, 2-ethylhexyl-(meth)acrylate, 2-ethoxyethyl(meth)acrylate, 2,3-dibromopropyl(meth)acrylate, 1,1-dihydroperfluorobutyl(meth)acrylate and any one mixture thereof. The term “(meth)acrylate” refers to both the acrylate as well as the corresponding methacrylate analog.

Another optical copolymer of interest is prepared from the sulfated lens monomer, TMPT3P or PTMC2P, a polythiol and one or more of the lens monomeric components above. The units of TMPT3P in the copolymer is present from 12 wt. % to 32 wt. %, preferably from 15 wt. % to 28 wt. %, and the units of polythiol from 15 wt. % to 40 wt. % preferably from 20 wt. % to 28 wt. %. The lens monomeric component is present from 15 wt % to 35 wt %. Again, the copolymer will also include one or more crosslink agents, and the units derived from such agents are present from 20 wt. % to 40 wt. %. In many embodiments of the optical copolymer, the crosslink agents will include polyethylene glycol (PEG) linkages.

The copolymer can further include a hydrophilic monomer component. The hydrophilic component is present in an amount from 2% to 20% by weight of the copolymer. Copolymers which include about 10% by weight or more of a hydrophilic monomeric component tend to form hydrogels if placed in an aqueous environment. Specific examples of useful hydrophilic monomeric components include N-vinyl pyrrolidone; hydroxyalkyl(meth)acrylates such as 2-hydroxyethyl(meth)acrylate, 3-hydroxypropyl(meth)acrylate, 3-hydroxypropyl(meth)acrylate, 4-hydroxybutyl(meth)acrylate, 2,3-dihydroxypropyl(meth)acrylate and the like; acrylamide; N-alkyl acrylamides such as N-methyl acrylamide, N-ethyl acrylamide, N-propyl acrylamide, N-butyl acrylamide and the like; acrylic acid; methacrylic acid; and the like and any one mixture thereof.

In one embodiment, the optical, polymeric materials can be prepared from one or more aromatic (meth)acrylate monomers having the formula:

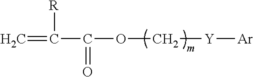

wherein: R is H or CH₃; m is an integer selected from 0 to 10; Y is nothing, 0, S, or NR wherein R is H, CH₃, C₂-C₆alkyl, iso-OC₃H₇, phenyl or benzyl; Ar is any aromatic ring, e.g., phenyl, which can be unsubstituted or substituted with H, CH₃, C₂H₅, n-C₃H₇, iso-C₃H₇, OCH₃, C₆H₁₁, Cl, Br, phenyl or benzyl; and a crosslinking component.

Exemplary aromatic (meth)acrylate monomers include, but are not limited to: 2-ethylphenoxy(meth)acrylate, 2-ethylthiophenyl(meth)acrylate, 2-ethylaminophenyl(meth)acrylate, phenyl-(meth)acrylate, benzyl(meth)acrylate, 2-phenylethyl(meth)acrylate, 3-phenylpropyl-(meth)acrylate, 4-phenylbutyl(meth)acrylate, 4-methylphenyl(meth)acrylate, 4-methylbenzyl(meth)acrylate, 2-2-methylphenylethyl(meth)acrylate, 2-3-methylphenylethyl(meth)acrylate, 2-4-methylphenylethyl(meth)acrylate, 2-(4-propylphenyl)ethyl(meth)acrylate, 2-(4-(1-methylethyl)phenyl)ethyl methacrylate, 2-(4-methoxyphenyl)ethyl methacrylate and the like.

Generally, if the optical, polymeric material is prepared with both an aromatic acrylate and an aromatic methacrylate as defined by the formula above, the materials will generally comprise a greater mole percent of aryl acrylate ester residues than of aryl methacrylate ester residues. It is preferred that the aryl acrylate monomers constitute from about 60 mol % to about 90 mol % of the polymer, while the aryl methacrylate monomers constitute from about 5 mol % to about 40 mol % of the polymer. Most preferred is a copolymer comprising about 50 mol % to 60 mol % 2-phenylethyl acrylate and about 15 mol % to 25 mol % 2-phenylethyl methacrylate.

In another embodiment, the optical, polymeric materials will have a fully hydrated (equilibrium) water content from 1.5% to 15% by weight, which also helps to minimize the degree of hazing or glistenings as described as well as minimize the formation of water vacuoles in vivo.

In another embodiment, the optical, polymeric material is prepared from sulfated lens monomer, which is present from 20% to 40% by weight; one of the aromatic lens monomer component above, which is present in 5% to 25% by weight; one of the alkyl lens monomer components above, which is present from 5% to 45%; and a hydrophilic monomer component, which is present from 20 to 60% by weight. Among the alkyl(meth)acrylates those containing 1 to 3 carbon atoms of alkyl group are preferred.

The optical, polymeric material can also be prepared by copolymerizing a specific monomer mixture comprising perfluorooctylethyloxypropylene(meth)acrylate, 2-phenylethyl(meth)acrylate, an alkyl(meth)acrylate monomer having the following general formula,

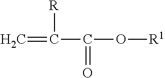

wherein R is hydrogen or methyl and R¹ is a linear or branched C₄-C₁₂ alkyl group, and a crosslinking monomer. An examplary list of alkyl(meth)acrylate monomer include n-butyl acrylate, isobutyl acrylate, isoamyl acrylate, hexyl acrylate, 2-ethylhexyl acrylate, octyl acrylate, isooctyl acrylate, decyl acrylate, isodecyl acrylate, and the like.

The perfluorooctylethyloxypropylene(meth)acrylate is present from 5% to 20% by weight, the 2-phenylethyl(meth)acrylate is present from 40% to 60% by weight, the alkyl(meth)acrylate monomer is present from 30% to 50% by weight and the crosslinking agent is present from 0.5% to 4% by weight.

The optical, polymeric component will likely include a crosslinking agent. The copolymerizable crosslinking agent(s) useful in forming the copolymeric material of the invention include any terminally ethylenically unsaturated compound having more than one unsaturated group. Preferably, the crosslinking agent includes a diacrylate or a dimethacrylate. The crosslinking agent may also include compounds having at least two (meth)acrylate and/or vinyl groups. Particularly preferred crosslinking agents include diacrylate compounds

The optical, polymeric materials are prepared by generally conventional polymerization methods from the respective monomeric components. A polymerization mixture of the monomers in the selected amounts is prespared and a conventional thermal free-radical initiator is added. The mixture is introduced into a mold of suitable shape to form the optical material and the polymerization initiated by gentle heating. Typical thermal, free radical initiators include peroxides, such as benzophenone peroxide, peroxycarbonates, such as bis-(4-t-butulcyclohexyl) peroxydicarbonate, azonitriles, such as azobisisobytyronitrile, and the like. A preferred initiator is bis-(4-t-butylcyclohexyl) peroxydicarbonate (PERK). Alternatively, the monomers can be photopolymerized by using a mold which is transparent to actinic radiation of a wavelength capable of initiating polymerization of these acrylic monomers by itself. Conventional photoinitiator compounds, e.g., a benzophenone-type photoinitiator, can also be introduced to facilitate the polymerization.

While specific embodiments of the present invention have been described in the foregoing, it will be appreciated by those skilled in the art that many equivalents, modifications, substitutions, and variations may be made thereto without departing from the spirit and scope of the invention as defined in the claims.

In one particularly useful embodiment, the copolymers are produced by mixing together the first component, the polythiol and the aromatic monomeric component (and optionally the hydrophilic monomeric component). The polymerization mixture, is well blended, deareated and poured into a mold. The mold is heated and the mixture is permitted sufficient time to cure. After curing the mold is disassembled and the molded optic recovered. Alternatively, the curing and post-curing occurs in a tube. The copolymer foamed in the tube is cut into cylindrical lens blanks. The lens blanks can be machined to produce the finished optic. Such machining may involve milling and lathing at cryogenic temperatures. Such polymerizations and manufacturing processes are well known to those in the art of making polymeric optical materials.

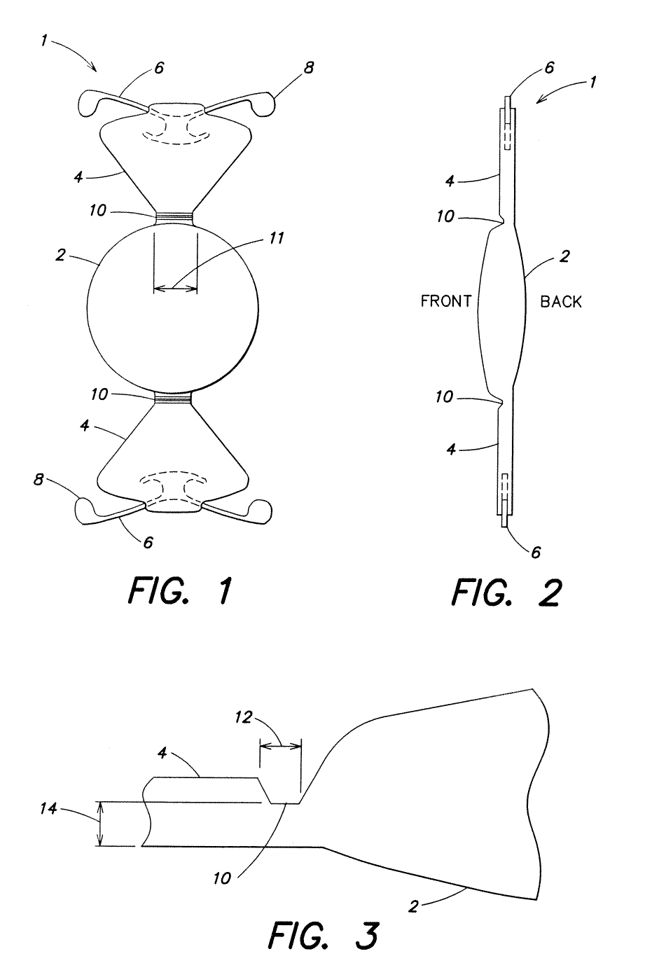

Turning now to the Figures, one embodiment of an accommodating intraocular lens is illustrated in FIGS. 1 to 3. An intraocular lens 1 formed as a flexible solid optic 2 made of the described copolymer, and flexible extending haptics 4 of any suitable form but preferably triangular plate haptics which are capable of multiple flexations without damage, and preferably, also formed of the copolymer. The optic 2 and haptics 4 preferably are uniplanar, and as shown two haptics 4 extend distally from opposite sides of the optic 2. Fixation and centration fingers 6 are provided at the distal ends of the haptics 4.

A typical length for the lens 1 is 10.5-11.5 mm, and the optic 2 typically is a 4.5 mm to 6.0 mm diameter optic. The fingers 6 preferably are approximately 5.0 mm wide and comprise four-point fixation loops that extend distally when the lens is put into any insertion cartridge. The ends 8 have a slightly different configuration and aid in indicating to the surgeon that the lens is right side up with the hinges in a proper position.

As shown, the haptics 4 have a triangular shape, with a narrow cross-section adjacent to the optic and wider at the outer ends. A hinge 10 is provided between the haptics 4 and the outer periphery of the optic 2, and it is particularly desirable to have a wide elastic base 10 to the hinge to allow the optic 2 to move forward more by stretching of the thin hinge base with the increase in vitreous cavity pressure, which can potentially provide more anterior movement of the lens. A typical hinge width 11 is 0.4 mm to 4.0 mm, and preferably with a hinge base width longitudinally as indicated by arrow 12 of 0.1 mm to 0.8 mm and preferably 0.5 mm, and a thickness range as indicated by arrow 14 of 0.06 mm to 0.4 mm, and preferably 0.12 mm, as indicated in FIG. 3. The wider hinge base stretches somewhat like an elastic band to facilitate greater anterior movement of the optic 2.

The hinges 10 are preferably on the anterior side as shown, and the round end 8 of loops 6 on the right as seen in FIG. 1 indicates that the hinge is uppermost. End 8 is round. The wider loops 6 minimize the anterior vault of the lens for distance vision and therefore provide better distance vision.

There can be a sharp edge around the posterior surface of the optic 2. To reduce the migration of cells across the posterior capsule of the lens post-operatively and thereby reduce the incidence of posterior capsular opacification and the necessity of YAG posterior capsulotomy.

A second embodiment of a described intraocular lens comprises haptics extending in a longitudinal direction between opposite portions of the equator of a capsular bag of an eye. The lens comprises an asymmetrical optic of substantially greater dimension transversely of said longitudinal direction and of lesser dimension in said longitudinal direction. The haptics extend oppositely longitudinally from the optic to engage the equator of the capsular bag. The haptics extend from capsular bag equator portions to attachment at opposite portions of the optic, whereby increased posterior vaulting of the optic is provided by the elongated haptics. The lens can also include an optic with a linear edge portions at longitudinally opposite sides with haptics being hinged to said opposite linear portions. The linear edge portions are indented from the periphery of the optic to enable elongation of the haptics. The lens may further include a light-transmitting skirt disposed about at least a portion of the periphery of the optic for reduction of glare impinging upon the retina of the eye.

Preferably, the optic has a longitudinal dimension of about 4.5 mm and a transverse dimension of about 6.0 mm. Also, the haptics can have transversely extending peripheral loop portions for engagement in the capsular bag equator.

Preferably, the haptics are hingedly mounted to the optic by flexible portions thereof adjacent to the optic. In one example, the haptics are hinged to the optic by grooved hinged portions of the haptics adjacent the optic.

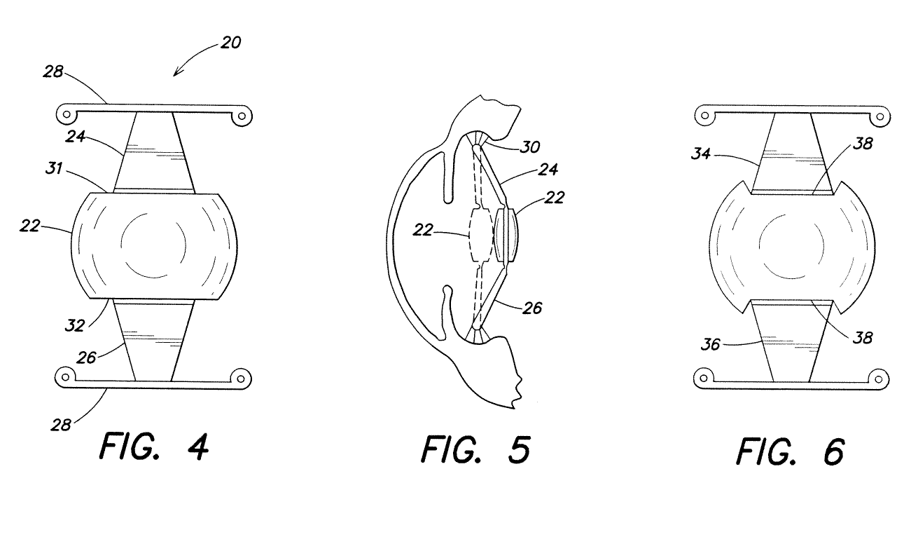

As illustrated in FIGS. 4 and 5, an accommodating lens 20 is shown as comprising an optic 22 and haptics 24, 26 extending oppositely therefrom and having loops 28 extending transversely thereof for engagement in the equator or rim of a capsular bag of an eye.

As shown, the lens is shortened in the longitudinal direction of haptics 24, 26 extension and elongated in the transverse direction, and the haptics are elongated in the longitudinal direction. From the geometry of the features and components, including the ciliary muscle 30, the haptics and the optic, it will be understood that the elongated haptics provide increased posterior vaulting of the optic, as indicated in FIG. 5. The optic thus has a somewhat oval configuration, with flat straight portions 31,32 hinged to the haptics. The lens provides improved, enhanced accommodation by increased posterior vaulting of the optic, while maintaining a maximal optical zone for accurate vision.

The optic 22, while relatively wide and enlarged in the direction transverse to the longitudinal direction of the haptics, and relatively short in the longitudinal direction, nevertheless has a full optical zone to provide full optical effect transmitted to the retina of the eye. Whereas artificial intraocular lenses typically have optical zones of less than 5.0 mm in diameter, particularly lenses with haptics staked into optics, the present invention provides optical zones of about 6.0 mm transversely and about 4.5 mm longitudinally.

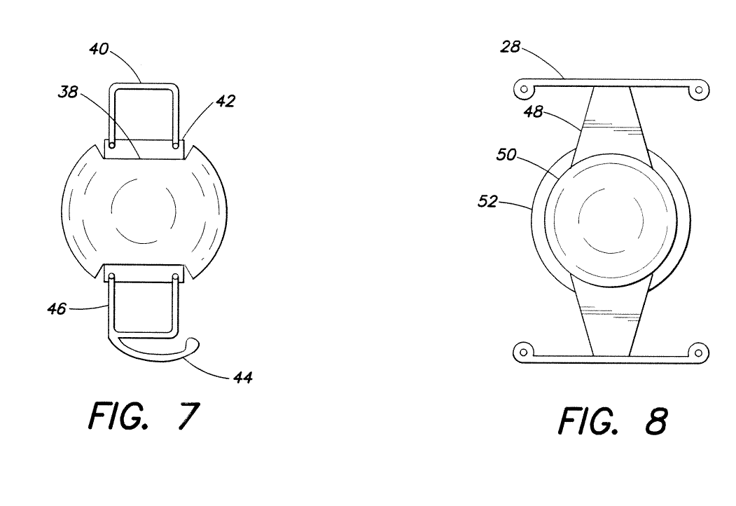

FIGS. 6 and 7 show embodiments of the invention wherein generally circular optics have indented linear portions 38, to which haptics 34, 36 are hingedly connected.

FIG. 7 shows a lens with indentations 38 at which are hingedly mounted haptics of generally rectilinear rod-like configuration, the haptics having plate elements 42 hingedly mounted to the optic. A loop haptic portion 44 extending transversely from an outer edge portion of a haptic 46 to aid in centering the lens within the capsular bag of the natural human lens. A haptic 40 is mounted on the other side of the lens.

FIG. 8 shows an embodiment wherein haptics 48 are hingedly mounted relative to an optic, and disposed about an optic 50 is a thin, annular transparent or translucent light-transmitting member 52 which reduces edge glare imposed on the retina.

As is well known in the art, an intraocular lens as described is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics, or loops, are positioned in the cul-de-sac of the capsular bag. The outer ends of the haptics, or the loops, are in close proximity with the bag cul-de-sac, and in the case of any form of loops, such as, the loops are deflected from the configuration. The ends or knobs of the loops are provided on the outer end portions of the loops for improved securement in the capsular bag or cul-de-sac by engagement with fibrosis, which develops in the capsular bag following the surgical removal of the central portion of the anterior capsular bag.

The inner ends of the loops may be either integrally formed from the same material as the haptics or the loops may be of a separate material such as polyimide. The loops if formed of a separate material are molded into the terminal portions of the haptics such that the flexible material of the loop can extend by elasticity along the internal fixation member of the loop.

The following non-limiting examples illustrate certain aspects of the present invention.

### Preparation of Thio-Monomers

### Example 1

### 3,6-dioxa-1,8-octane di(3-isopropenyl-α,α-dimethyl benzyl thiocarbamate)

(DOIDBTC); 2759-154

### Components:

3-isopropenyl-α,α-dimethyl benzyl isocyanate (IDBI)
Triethyl amine (TEA))
2,6 di-tert-butyl-4 methylphenol (BHT)
3,6 dioxa-1,8 octanedithiol (DODT)
anhydrous dichloromethane

### Procedure:

A flame dried 500 mL 3-neck RB flask is fitted with a 125 mL addition funnel, Friedrich's condenser with nitrogen inlet, magnetic stirrer, temperature probe, and heating mantle. IDBI (20.8 mL, 0.1 mol), TEA (1.0 mL, 0.0072 mol), BHT (29.1 mg, 2.28×10⁻⁴ mol), and 125 mL CH₂Cl₂ are added to the flask and allowed to mix under a dry nitrogen atmosphere. The flask is heated to 40° C. and allowed to equilibrate. The addition funnel is loaded with DODT (8.2 mL, 0.05 moles) and 50 mL CH₂Cl₂ is purged with dry nitrogen. This solution is added dropwise over one hour to the stirred reaction components. The reaction is stirred for 36 hrs at a temperature control of 40° C. The solvent is removed under pressure at ambient temperature. Expected yield: 29.24 g (actual % yield 99+, quantitative reaction).

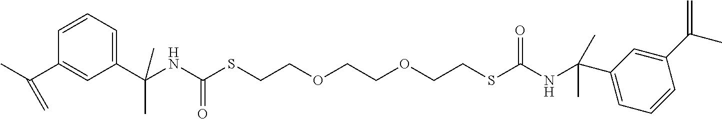

### Example 2

### Synthesis of IEM-PETMA; 2759-144

The synthetic procedure of example 1 is used with the following modifications. 2-isocyanatoethylmethacrylate (IEM) (14.4 mL, 0.102 mol), TEA ((0.150 μL, 0.0011 mol) and BHT (30.2 mg, 10.3×10⁻⁴ mol) and 50 mL CH₂Cl₂ are added to the rb flask, allowed to mix under a dry nitrogen and heated to 40° C. Pentaerythritol tetrakis(3-mercapto-proprionate) (PETMP) (9.7 mL, 0.025 mol) and 125 mL CH₂Cl₂ are then added dropwise over one hour to the stirred reaction components. The reaction is allowed to proceed for an additional 24 hrs at 40° C. The solvent is removed under pressure at ambient temperature. Expected yield: 27.74 g (actual % yield 99+, quantitative reaction).

### Example 3

### Pentaerytritrol tetrakis[3-mercapto cabamoyl isopropecryl 3-isopropene benzene (PTMC2B) 2913-6

The synthetic procedure of example 1 is used with the following modifications. IDBI (42.6 mL, 0.215 mol), TEA (2.0 mL, 0.027 mol) and BHT (55.2 mg, 2.5×10⁻⁴ mol) and 250 mL CH₂Cl₂ are added to the rb flask, allowed to mix under a dry nitrogen and heated to 40° C. PETMP (20.2 mL, 0.053 mol) and 100 mL CH₂Cl₂ are then added dropwise over one hour to the stirred reaction components. The reaction is allowed to proceed for an additional 48 hrs at 40° C. The solvent is removed under pressure at ambient temperature. Yield: 66.93 g (97.8% by weight)

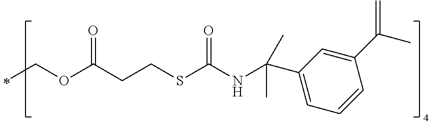

### Examples 4 to 6

TABLE 1

| Parts of Polymer Component | Ex. 4 | Ex. 5 | Ex. 6 |
|---|---|---|---|
| PETMP | 44.3 | 50.5 | 44.9 |
| TMPTMA | 39.3 | 47.2 | 22.4 |
| PEG 600 DMA | 13.9 | — | — |
| TPP | 1.5 | 1.3 | 1.2 |
| TEGDMA | — | — | 30.5 |
| Darocure 1173 | 1 | 1 | 1 |

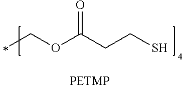

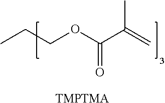

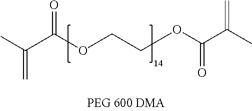

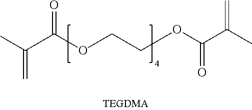

For each of Examples 4 to 6 a polymer mix that included all of the polymer components of Table 1 with the exception of PETMP was prepared. The mixture was stirred until a homogeneous mixture was observed. The PETMP was added to the homogeneous mixture. The mix was placed into a vacuum chamber and degassed until no more bubbles were observed coming from the mixture, typically 10-15 minutes. The polymer mix was placed between two silanated glass plates, and teflon tape was applied in layers around the edges of one plate leaving a space for excess mix to escape until a desired thickness was achieved. Binder clips were used to keep the plates from separating. The mix was then cured under UV light over 2 hrs under nitrogen. Mercury fluorescent lamps with emission maximum at 365 nm, and an intensity 7-12 mW/cm² was used. The hard film was then extracted using 30% Ethanol overnight. The mechanical properties of the resulting film polymers are provided in Table 1B.

TABLE 1B

| Properties | Ex. 4 | Ex. 5 | Ex. 6 |
|---|---|---|---|
| modulus (g/cm²) | 6747 | 9077 | 2045 |
| tensile (g/cm²) | 693 | 935 | 356 |
| % elongation | 15 | 11 | 19 |
| tear | 23 | brittle | 6 |
| water content % | 3.4 | 2.4 | 2.7 |
| refractive index (546 nm) | 1.53 | 1.54 | 1.54 |

### Examples 7 to 10

TABLE 2

| Parts of Polymer | | | | |
|---|---|---|---|---|
| Component | Ex. 7 | Ex. 8 | Ex. 9 | Ex. 10 |
| TMPTMP | 32.7 | 22.8 | 16.4 | 39.1 |
| TMPT3P | 22.9 | 32.7 | 39.5 | 16.9 |
| PEG 600 DMA | 32.9 | 33 | 32.6 | 32.6 |
| TEGDMA | 9.8 | 9.8 | 9.8 | 9.8 |
| TPP | 1 | 1 | 1 | 1 |
| Vazo 67 | 0.65 | 0.65 | 0.65 | 0.65 |

For each of Examples 7 to 10 a polymer mix that included all of the polymer components of Table 2 with the exception of TMPTMP was prepared. The mixture was stirred until a homogeneous mixture was observed. The TMPTMP was added to the homogeneous mixture. The mix was placed into a vacuum chamber and degassed until no more bubbles were observed coming from the mixture. Typically 10-15 minutes. The polymer mix was placed between two silanated glass plates, and teflon tape was applied in layers around the edges of one plate leaving a space for excess mix to escape until a desired thickness was achieved. Binder clips were used to keep the plates from separating. The film was cured thermally using the Award Cycle in a Blue M oven. The cure schedule was as follows:

a. 30 minute nitrogen purge

b. Ramp to 65 C

c. Hold at 65 C for 19 minutes

d. Ramp to 93 C

e. Hold at 93 C for 30 minutes

f. Ramp to 110 C

g. a Hold at 110 C for 59 minutes

h. Cool

The hard film was then extracted using 30% ethanol overnight.

TABLE 2B

| Properties | Ex. 7 | Ex. 8 | Ex. 10 |
|---|---|---|---|
| modulus (g/cm²) | 328 | 622 | 180 |
| tensile (g/cm²) | 73 | 97 | 63 |
| % elongation | 28 | 20 | 45 |
| tear | 2 | 3 | 2 |

### Examples 11 to 14

TABLE 3

| Parts of Polymer | | | | |
|---|---|---|---|---|
| Component | Ex. 11 | Ex. 12 | Ex. 13 | Ex. 14 |
| PTMC2B | 23.3 | 22.9 | 23.4 | 23.9 |
| PETMP | 32.7 | 33 | 32.7 | 33.8 |
| PEG 600 DMA | 9.8 | 16.3 | 32.5 | 3.4 |
| TEGDMA | 32.6 | 26.1 | 9.8 | 37.2 |
| TPP | 1 | 1 | 1 | 1 |
| Darocure 1173 | 0.65 | 0.65 | 0.65 | 0.65 |

TABLE 3B

| Properties | Ex. 11 | Ex. 12 | Ex. 13 | Ex. 14 |
|---|---|---|---|---|
| modulus (g/cm²) | 906 | 794 | 576 | 899 |
| tensile (g/cm²) | 239 | 220 | 150 | 231 |
| % elongation | 35 | 36 | 31 | 32 |
| tear | 17 | 7 | 3 | 26 |
| water content % | 3.4 | 4.4 | 10.2 | 3 |

TABLE 4

| Parts of Polymer | | | | | |
|---|---|---|---|---|---|
| Component | Ex. 15 | Ex. 16 | Ex. 17 | Ex. 18 | Ex. 19 |
| PTMC2B | 23.1 | 23.2 | 23.1 | 22.9 | 22.9 |
| PETMP | 22.9 | 16.4 | 22.8 | — | — |
| DPDBMP | 9.9 | 16.3 | 9.9 | 32.8 | 32.8 |
| PEG 600 DMA | 9.8 | 9.8 | 10 | 9.8 | 9.8 |
| TEGDMA | 32.7 | 32.7 | 32.6 | 32.8 | 32.8 |
| TPP | 1 | 1 | 1 | 1 | 1 |
| Vazo 67 | 0.65 | 0.65 | 0.65 | 0.66 | 0.66 |

TABLE 4B

| Properties | Ex. 15 | Ex. 16 | Ex. 17 | Ex. 18 | Ex. 19 |
|---|---|---|---|---|---|
| modulus (g/cm²) | 621 | 566 | 661 | 407 | 613 |
| tensile (g/cm²) | 78 | 88 | 120 | 79 | 144 |
| % elongation | 16 | 18 | 25 | 23 | 32 |
| tear | 3 | 4 | 4 | 3 | 3 |
| water content % | 4.2 | 4.2 | 4 | 5 | 3.7 |

### Example 20

### Variable Polymerization Conditions

The components reported as Example 11 (minus initiator) were subjected to various polymerization reaction conditions using different initiators and concentrations as reported in Table 5. A photocure of two hours was used for A thru E. Sample F was a thermal cure.

TABLE 5

| Example 11 | A | B | C | D | E | F |
|---|---|---|---|---|---|---|
| initiator | Daro 1173 | Irga 819 | Irga 819 | Irga 819 | Lucirin | Vazo 67 |
| conc. | 0.66 | 0.66 | 1.67 | 2.53 | TPO-L | 0.95 |
| (wt. %) | | | | | 0.66 | |
| cure | UV | visible | visible | visible | visible | thermal |
| condition | | | | | | |
| modulus | 847 | 915 | 859 | 915 | 816 | 775 |
| (g/cm²) | | | | | | |
| tensile | 194 | 235 | 211 | 209 | 210 | 193 |
| (g/cm²) | | | | | | |
| % elongation | 27 | 32 | 29 | 30 | 29 | 29 |
| tear | 20 | 12 | 13 | 13 | 15 | 8 |
| Tg ° C. | −3.3 | −10.5 | −9.3 | −8.9 | −4.6 | −9.8 |

We claim:

1. A sulphated lens monomer of formula I
[PG-R¹—NHC(Y)S—R²]ₙ—R³ I
wherein PG is a polymerizable group selected from the group consisting of vinyl, acryloyl, acryoyloxy, methacryoyl and methacryoyloxy; acrylamido and methacrylamido; Y is oxygen or sulfur, and n is 2, 3 or 4;
R¹ is an aliphatic group of one to eight carbon atoms and optionally with one or more oxygen atoms or an aromatic ring;
R² is a bond or an organic group of one to six carbon atoms; and
R³ is a bond, an organic group of one to ten carbons atoms, carbon, oxygen, sulfur or a disulfide, wherein the lens monomer is derived from a first component of formula,
PG-R¹—NC(Y)
and a polythiol of formula, R³—[R²—SH]ₙ.

2. The lens monomer of claim 1 wherein R³ is oxygen, sulfur or disulfide and R² is alkylene with two to four carbons or comprises a phenyl group.

3. The lens monomer of claim 1 wherein R³ is a bond, a carbon or —CH and R² is alkylene with one to three carbons.

4. The lens monomer of claim 1 wherein R³—[R²—SH]ₙ is selected from one of the following polythiols.

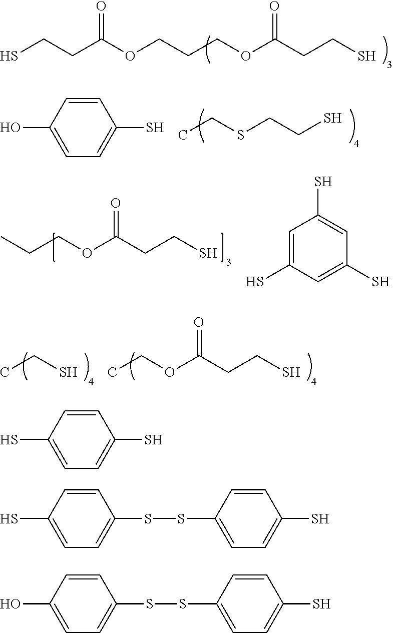

5. The lens monomer of claim 1 wherein R¹ is alkylene with one to three carbons or comprises a phenyl group.

6. The lens monomer of claim 1 wherein R¹ includes an aromatic ring; R² is alkylene with two to four carbons; R³ is carbon, ethyl or propyl; and Y is oxygen.

7. The lens monomer of claim 1 of formula II 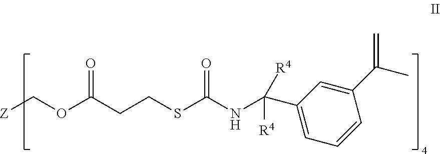
wherein Z is carbon, or an alkyl with two to four carbons and R⁴ is independently selected from hydrogen or methyl.

8. An optical copolymer comprising:
a first constituent derived from a first component of general formula III
PG-R—NCY III
wherein PG is a polymerizable group selected from the group consisting of vinyl, acryloyl, acryoyloxy, methacryoyl and methacryoyloxy; acrylamido and methacrylamido; R is an organic group of one to eight carbon atoms and optionally with one or more oxygen atoms or an aromatic ring; and Y is oxygen or sulfur;
a second constituent derived from a polythiol; and
a third constituent derived from an aromatic monomeric component, the homopolymer of which has a refractive index of at least about 1.48.

9. The copolymer of claim 8 is optically clear and has a refractive index of at least about 1.51.

10. The copolymer of claim 9 having an equilibrated water content of 1.5% to 15%.

11. The copolymer of 10 wherein the aromatic monomeric component is selected from the group consisting of styrene, vinyl carbazole, vinyl naphthalene, benzyl acrylate, phenyl acrylate, naphthyl acrylate, pentabromophenyl acrylate, 2-phenoxyethyl acrylate, 2-phenoxyethyl methacrylate and any one mixture thereof.

12. A copolymer comprising:
a constituent derived from lens monomer of general formula I
[PG-R¹—NHC(Y)S—R²]ₙ—R³ I
wherein PG is a polymerizable group selected from the group consisting of vinyl, acryloyl, acryoyloxy, methacryoyl and methacryoyloxy; acrylamido and methacrylamido; Y is oxygen or sulfur, and n is 2, 3 or 4;
R¹ is an aliphatic group of one to eight carbon atoms and optionally with one or more oxygen atoms or an aromatic ring;
R² is a bond or an organic group of one to six carbon atoms; and
R³ is a bond, an organic group of one to ten carbons atoms, carbon, oxygen, sulfur or a disulfide, wherein the lens monomer is derived from a first component of formula, PG-R¹—NC(Y), and a polythiol of formula, R³—[R²—SH]ₙ.

13. The copolymer of claim 12 is optically clear and has a refractive index of at least about 1.51.

14. The copolymer of claim 13 having an equilibrated water content of 1.5% to 5%.

15. The copolymer of claims 13 further comprising a constituent derived from an aromatic monomeric component, the homopolymer of which has a refractive index of at least about 1.48.

16. The copolymer of claim 15 wherein the aromatic monomeric component is selected from the group consisting of styrene, vinyl carbazole, vinyl naphthalene, benzyl acrylate, phenyl acrylate, naphthyl acrylate, pentabromophenyl acrylate, 2-phenoxyethyl acrylate, 2-phenoxyethyl methacrylate and any one mixture thereof.

17. The copolymer of claim 14 wherein R³ is oxygen, sulfur or disulfide and R² is methylene with two to four carbons or phenyl.

18. The copolymer of claim 14 wherein R³ is a bond, a carbon or —CH and R² is methylene with one to three carbons.

19. The copolymer of claim 12 wherein R¹ is methylene with one to three carbons or comprises a phenyl group.

20. The copolymer of claim 12 wherein R³—[R²—SH]ₙ is selected from one of the following polythiols.

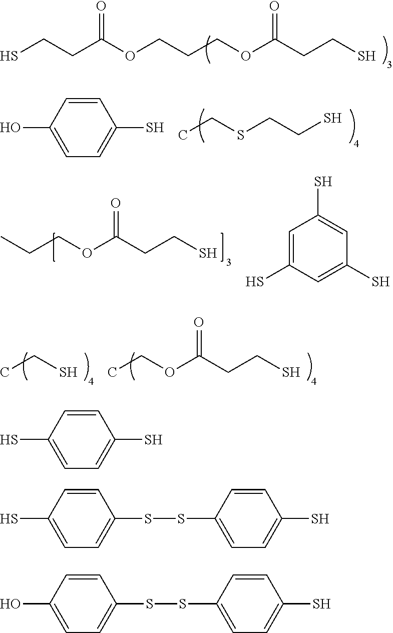

21. The copolymer of claim 12 further comprising a constituent derived from a crosslink agent that includes a polyethylene glycol linkage.

22. An ophthalmic lens comprising a copolymer of claim 8.

23. The ophthalmic lens of claim 22 wherein the lens is an intraocular lens.
